(19) United States
(12) Patent Application Publication
(10) Pub. No.: US 2013/0040395 A1
(43) Pub. Date: Feb. 14, 2013

(54) USE OF HALOGENATED DERIVATIVES OF THE CYANOCINNAMIC ACID AS MATRICES IN MALDI MASS SPECTROMETRY

(71) Applicants: Thorsten Jaskolla, Weiterstadt (DE); Michael Karas, Hattersheim (DE)

(72) Inventors: Thorsten Jaskolla, Weiterstadt (DE); Michael Karas, Hattersheim (DE)

(73) Assignee: JOHANN WOLFGANG GOETHE-UNIVERSITÄT, Frankfurt am Main (DE)

(21) Appl. No.: 13/582,574

(22) PCT Filed: Mar. 2, 2011
(86) PCT No.: PCT/DE2011/000204

§ 371 Date: Oct. 25, 2012

(30) Foreign Application Priority Data
Mar. 4, 2010 (DE) 10 2010 010 155.9

(51) Int. Cl. C07C 255/41; G01N 27/62

(52) U.S. Cl. 436/86; 558/300

(57) ABSTRACT

The present invention relates to the use of a halogenated cyanocinnamic acid derivative with the general formula:

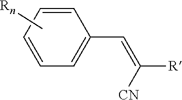

wherein R is selected independently among F, Cl and Br, n=3, 4 or 5 and R′ is selected among COOH, CONH₂, SO₃H and COOR″ with R″=C₁-O₅-Alkyl; and/or of 4-bromo-α-cyanocinnamic acid and/or of α-cyano-2,4-dichlorocinnamic acid in a matrix for a MALDI mass spectrometry of an analyte.

The present invention relates to the use of an halogenated cyanocinnamic acid derivative as a matrix in MALDI mass spectrometry of analytes as well as to such a matrix.

MALDI (Matrix-Assisted Laser Desorption/Ionization) is a technology for ionization of different molecule classes developed in the early 1980s. This method is based on incorporation of the analytes, which are to be analyzed, in an organic matrix by means of co-crystallization. The subsequent irradiation of the co-crystallized sample with a laser causes energy input through absorption of the matrix and serves for the desorption of individual molecules and molecule agglomerates in the gas phase necessary for the analysis as well as for their fragmentation to the corresponding monomolecular species. Furthermore, the laser energy causes (photo)ionization of the matrix and the analytes and permits in this way their separate detection by accelerating the formed ions in electromagnetic fields and measuring the time-of-flight depending on the mass and the charge. By the incorporation of the analytes in a large molar overflow in the matrix, their excessive fragmentation is prevented, so that the MALDI method is suitable not only for detection of smaller molecules, such as medicinal substances, metabolites or peptides, but also in particular well suited for detection of intact large and thermally unstable biomolecules, such as proteins, oligonucleotides, or also, for example, of synthetic polymers or macromolecular inorganic compounds.

Mostly small organic molecules, which have a sufficiently strong absorption capacity at the used laser wavelengths (mostly UV or IR lasers), are used as matrices. In addition, various extra matrix requirements must be fulfilled, e.g. efficient incorporation of analytes in the matrix by means of co-crystallization with it, separation of the analytes within the matrix crystal, vacuum stability, solvability in an analyte-compatible solvent and high analyte sensitivities. The common matrices have unstable protons as in carboxylic acids or acidic hydroxyl groups. The best-known matrix compound is the mostly used α-cyano-4-hydroxycinnamic acid (CHCA or HCCA). Other matrices used in the MALDI-MS are, for example, the 4-chloro-α-cyanocinnamic acid (ClCCA), the sinapic acid (4-hydroxy-3,5-dimethoxycinnamic acid) or the 2,5-dihydroxybenzoic acid (2,5-DHB). Independently of their specific structure, the matrices can be used practically for all substances that can be analyzed with the MALDI-MS, which include, among others, large and small as well as non-volatile and thermally unstable compounds, with biomacromolecules such as proteins and lipids among them, as well as organic and inorganic analytes such as medicinal substances, plant metabolites and the like. The current analytical focal point is in the analysis of peptides.

The use of hydroxy- or methoxy-substituted cinnamic acids as matrices in the MALDI mass spectrometry is known from Beavis, R. C.; Chait, B. T. “Cinnamic Acid Derivatives as Matrices for Ultraviolet Laser Desorption Mass Spectrometry of Proteins”, Rapid Communication in Mass Spectrometry (1989), Vol. 3, No. 12, pp. 432-435. The corresponding use of the α-cyanocinnamic acid results from US 2002/0142982 A1. DE 101 58 860 A1 and DE 103 22 701 A1 describe the use of the α-Cyano-4-hydroxycinnamic acid or the 3,5-dimethoxy-4-hydroxycinnamic acid. The use of the halogenated a-cyanocinnamic acid is described in DE 10 2007 040 251 A1, the use of the ClCCA al a MALDI matrix is known from Jaskolla et al. “4-Chloro-α-cyanocinnamic acid is an advanced, rationally designed MALDI matrix”, Proc. Natl. Acad. Sci. USA (2008), Vol. 105, No. 34, pp. 12200-12205. Furthermore, the US 2006/0040334 A1 and WO 2006/124003 A1 disclose MALDI matrices on the basis of analyte-coupled or polymer-coupled derivatives of the α-cyano-4-hydroxycinnamic acid.

Various advantages for the mass-spectrometric analyses can be obtained by means of sensitive detection of deprotonated analytes in the negative ion mode. So by using complementary measurements of both the positive and negative ion mode, additional information about the analyte properties such as, for example, analyte acidity, can become accessible; by using additional signals, which cannot be detected in the positive ion mode, enhanced sequence coverage, for example, of peptides and proteins can be achieved, or by analysis of analyte fragments with negative charge additional structural information can be obtained in the process of fragmentation. In addition, due to missing base groups and/or the presence of easily deprotonable functions, various analyte classes can be only protonated with difficulty, such as, for example, phosphotyrosine-containing analytes (Bonewald et al. “Study on the synthesis and characterization of peptides containing phosphorylated tyrosine”, Journal of Peptide Research (1999), Vol. 53, No. 2, pp. 161-169), sulfated analytes (Nabetani et al. “Analysis of acidic peptides with a matrix-assisted laser desorption/ionization mass spectrometry using positive and negative ion modes with additive monoammonium phosphate”, Proteomics (2006), Vol. 6, pp. 4456-4465), chlorated lipids with small base dichloramine headgroups (Jaskolla et al. “The new matrix CICCA allows the detection of phosphatidylethanolamine chloramines by MALDI-TOF MS”, Journal of the American Society for Mass Spectrometry (2009), Vol. 20, pp. 867-874) or strongly acid peptides (Jainhuknan, J. and Cassady, C. J. “Negative ion postsource decay time-of-flight mass spectrometry of peptides containing acidic amino acid residues”, Analytical Chemistry (1998), Vol. 70, No. 24, pp. 5122-5128). This goes along with reduced sensitivities in the positive ion mode and in connection with the aforementioned possibilities for additional information gains the need for sensitive matrices for the negative ion mode.

The matrix compounds, which were known until now, are almost exclusively suitable only for the detection of ions with a positive charge. Improvements as well as rational approaches aim predominantly to optimizations of the sensitivity in this polarity. Consequently, the positive ion mode is used approximately quantitatively for analytical issues. Compared to the corresponding positive ion measurements, the measurements in the negative ion mode often show reduction of the analyte signal intensities by several orders of magnitude and are thus comparatively sporadically described in the scientific literature. The few known approaches with a sensitivity increase in the negative ion mode are limited mostly to a variation in the preparation conditions and/or device parameters such as, for example, measurements in a linear mode, in which higher sensitivities are achieved by a drastic reduction of the resolution (Janek et al. “Phosphopeptide analysis by positive and negative ion matrix-assisted laser desorption/ionization mass spectrometry”, Rapid Communication in Mass Spectrometry (2001), Vol. 15, pp. 1593-1599).

The obtainable sensitivities of the MALDI mass spectrometry for the detection and proof sensitivity of ions with a positive and negative charge are currently unsatisfactory and need further improvements.

Therefore, the task of the present invention is to provide compounds by optimization of the matrix structure, which compounds at least partially overcome the disadvantages of the current state of the art for the different analytes, among them the mostly studied class of the peptides, by significantly increasing the sensitivities of the analytes in the positive and negative ion modes.

This task is solved by the use of a halogenated cyanocinnamic derivative with the general formula:

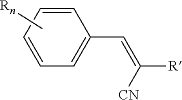

wherein R is selected independently among F, Cl and Br, n=3, 4 or 5 and R′ is selected among COOH, CONH₂, SO₃H and COOR″ with R″=C₁-C₅-alkyl; and/or of 4-bromo-α-cyanocinnamic acid and/or of α-cyano-2,4-dichlorocinnamic acid in a matrix for a MALDI mass spectrometry of an analyte.

According to the invention, both cis-isomers and trans-isomers of the halogenated cyanocinnamic acid derivatives can be used. Constitution isomers of the halogenated cyanocinnamic acid derivatives can also be used.

Since only three or four halogen substituents are available in the phenyl ring, the remaining phenyl substituents are hydrogen. The structural formula shown above should comprise all possible configuration isomers (cis/trans-isomers) and constitution isomers (positional isomers) of the different cyanocinnamic acid derivatives. So, for example, a cyanocinnamic acid derivative, which is substituted in the positions 2, 3 and 4 of the phenyl ring, should correspond to the cyanocinnamic acid derivative, which is substituted with the same substituents in the positions 4, 5 and 6. The selection of F, Cl and Br as possible substituents is completely independent from each other; each R can be selected by a different one.

In a particularly preferred embodiment, R is selected among F, Cl and Br with n=5 and R′=COOH. Exemplary preferred compounds are α-cyano-2,3,4,5,6-pentafluorocinnamic acid, α-cyano-2,3,4,5,6-pentachlorocinnnamic acid and α-cyano-2,3,4,5,6-pentabromocinnamic acid.

In another preferred embodiment, R is selected among F, Cl and Br with n=4 and R′=COOH. Compounds preferred here comprise α-cyano-2,3,4,5-tetrafluorocinnamic acid, α-cyano-2,3,4,5-tetrachlorocinnamic acid and α-cyano-2,3,4,5-tetrabromocinnamic acid.

Furthermore, the cyanocinnamic acid derivative can be preferably selected among α-cyano-2,3,5,6-tetrafluorocinnamic acid, α-cyano-2,3,5,6-tetrachlorocinnamic acid and α-cyano-2,3,5,6-tetrabromocinnamic acid.

Furthermore, preferred are also cyanocinnamic acid derivatives, which are selected among α-cyano-2,4,5,6-tetrafluorocinnamic acid, α-cyano-2,4,5,6-tetrachlorocinnamic acid and α-cyano-2,4,5,6-tetrabromocinnamic acid.

It is also proposed preferably that R is selected among F, Cl, Br with n=3 and R′=COOH. Exemplary preferred compounds are a-cyano-2,3,4-trifluorocinnamic acid, α-cyano-2,3,4-trichlorocinnamic acid, α-cyano-2,3,4-tribromocinnamic acid, α-cyano-2,3,5-trifluorocinnamic acid, α-cyano-2,3,5-trichlorocinnamic acid, α-cyano-2,3,5-tribromocinnamic acid, α-cyano-2,3,6-trifluorocinnamic acid, α-cyano-2,3,6-trichlorocinnamic acid, α-cyano-2,3,6-tribromocinnamic acid, α-cyano-2,4,5-trifluorocinnamic acid, α-cyano-2,4,5-trichlorocinnamic acid, α-cyano-2,4,5-tribromocinnamic acid, α-cyano-2,4,6-trifluorocinnamic acid, α-cyano-2,4,6-trichlorocinnamic acid, α-cyano-2,4,6-tribromocinnamic acid, α-cyano-3,4,5-trifluorocinnamic acid, α-cyano-3,4,5-trichlorocinnamic acid and α-cyano-3,4,5-tribromocinnamic acid.

Another also particularly preferably used α-cyanocinnamic acid is the 4-brom-α-cyanocinnamic acid and/or α-cyano-2,4-dichlorocinnamic acid.

It is then preferred that the matrix is used for MALDI mass spectrometry negative ions.

Particularly preferred is n=5.

In one embodiment, the analyte can be selected among protein, peptide, polynucleic acid, lipid, phosphorylated compound, saccharide, medicinal substance, metabolite, synthetic and natural (co)-polymer and inorganic compound. Exemplary analytes are, among others, the phosphopeptides or phospholipides, dendrimeres and polynucleic acids, for example DNA, RNA, siRNA, miRNA.

It is preferably proposed also that the matrix is mixed with the analytes.

The molar mixing ratio of analyte to matrix can then be from 1:100 to 1:1000000000, preferably 1:10000.

Also mixtures of halogenated cyanocinnamic acid derivatives, which belong to the above formula, can be used.

According to the invention, the most preferred for use is a mixture of at least one of the aforementioned halogenated cyanocinnamic acid derivatives with at least one additional matrix material.

The additional matrix material can preferably be selected among the α-cyano-4-hydroxycinnamic acid, α-cyano-2,4-difluorocinnamic acid, 2,5-dihydroxybenzoic acid, sinapic acid, ferulic acid, 2-aza-5-thiothymine, 3-hydroxypicolinic acid and 4-chlor-α-cyanocinnamic acid.

When mixtures with other matrix materials are used in accordance with the invention, the additional matrix materials are used in a ratio from 10 to 90 weight percent, preferably 20 to 50 weight percent, related to the total weight of the matrix materials.

In another preferred embodiment, the matrix material is mixed with an inert filler.

The matrix can be available also as ionic liquid.

Furthermore is also in accordance with the invention a matrix for a MALDI mass spectrometry of an analyte, which comprises a halogenated cyanocinnamic acid derivative with the general formula:

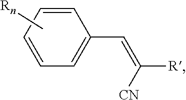

wherein R is selected independently among F, Cl and Br, n=3, 4 or 5 and R′ is selected among COOH, CONH₂, SO₃H and COOR″ with R″=C₁-C₅-alkyl; and/or comprises 4-bromo-α-cyanocinnamic acid and/or α-cyano-2,4-dichlorocinnamic acid.

In a preferred embodiment, the matrix is available as ionic liquid. For the production of an ionic liquid, the matrix can be mixed with protonatable bases such as, for example, pyridine, diethylamine or 3-aminoquinoline, whereby an ionic matrix solution is formed, which is mixed with analyte solutions or is applied as a film on surfaces to be examined.

In another embodiment, the matrix can be solved in a solvent with low vapor pressure, for example glycerin, and is then mixed with solved analytes or is applied as a film on surfaces to be examined.

For performing the MALDI mass spectrometry, preferably an IR laser, UV laser, such as Nd:YAG or nitrogen laser, as well as lasers emitting in the midrange or far-range UV, such as a tunable color or optic parametric oscillator (OPO) laser and a 308 nm XeCl excimer laser, is used for energy input.

Surprisingly, it was established, according to the invention, that in the use of multiply halogenated α-cyanocinnamic acid derivatives in a matrix in the MALDI mass spectrometry, the detection and the detection sensitivity for positive and negative ions, preferably negative ions, can be significantly improved. The use of the derivatives leads to clearly stronger analyte signal intensities and sensitivities in the negative ion mode than previous matrices.

The use of matrix mixtures according to a preferred embodiment permits to optimize the properties with respect to the crystallization and the incorporation of analyte compounds in the matrix crystals as well as to achieve more efficient ionization of the analytes.

Without preferring to be bound to any given theory, it is assumed that the advantages, based on the selection of electron-pulling as well as lightly polarizable substituents, exist independently of the exact positioning and number of the halogen substituents in the aromatic compounds.

It is assumed that the following advantages can be achieved by the use of the proposed halogenated α-cyanocinnamic acid derivatives according to the invention: thanks to the pronounced -I-effect of very active electron-attracting halogens, the highly negative charge density of the matrix carbanions formed during the secondary ionization process is reduced and thus their stability is increased; in the neutral matrices, the electron-attracting substituents cause a reduction of the negative charge density of the respective matrix-π-systems, whereby the electrons, which are freed during the photoionization of the matrix or the metallic substrate and are also negatively charged, have to overcome a lower repulsion force for the absorption by other neutral matrix molecules, which results in higher electron affinity and capturing probability, respectively; when the electron affinity increases through the derivatization with halogen substituents, then there is more internal energy for the formation of reactive carbanions.

Additional features and advantages of the invention result from the following detailed description of preferred embodiments in connection with the enclosed drawings, in which:

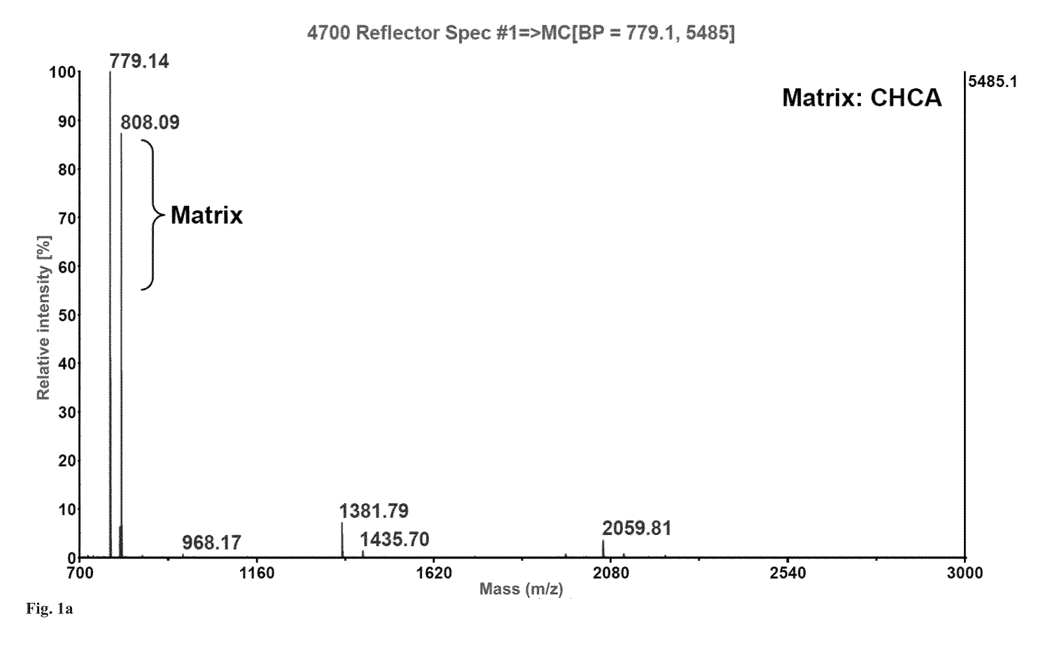

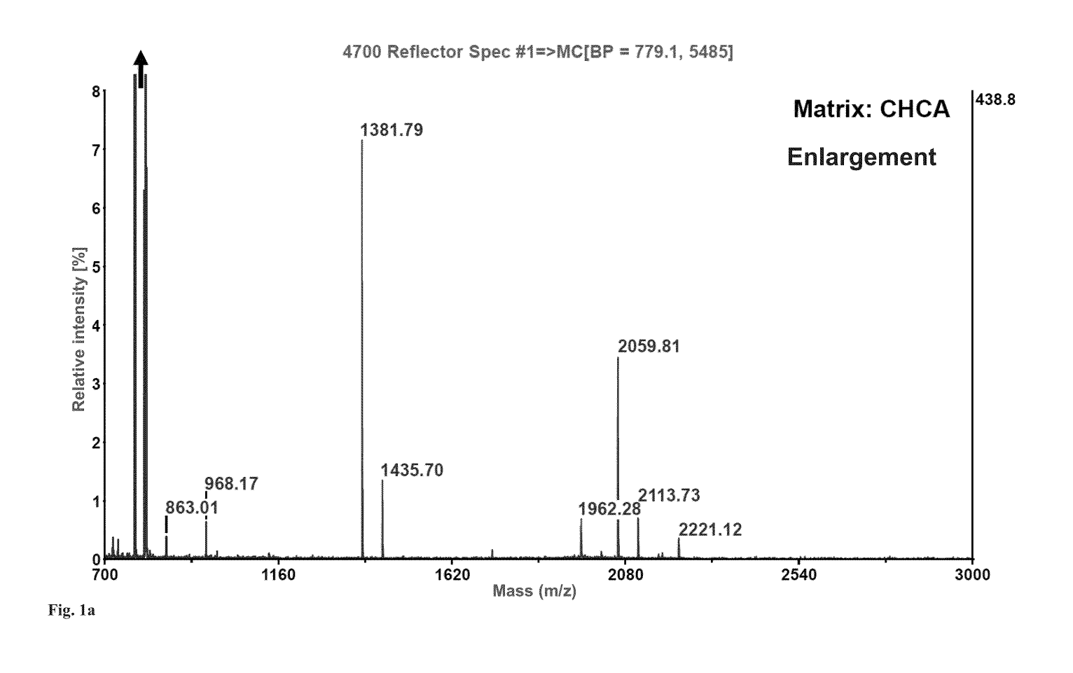

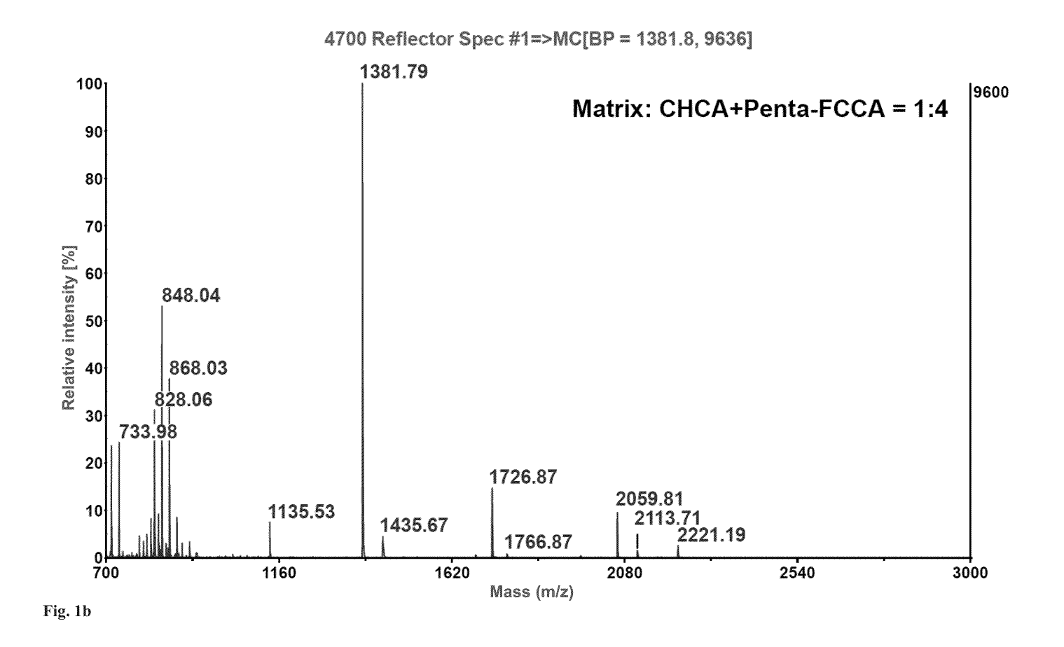

FIG. 1a shows a section (top) of the mass spectrum recorded in the negative ion mode of a tryptic β-casein digestant obtained with CHCA as well as a corresponding enlargement (bottom) on the analyte fragments, and FIG. 1b shows the corresponding section of a mass spectrum recorded in the negative ion mode of a tryptic β-casein digestant obtained by using a matrix mixture CHCA+α-cyano-2,3,4,5,6-pentafluorocinnamic acid (penta-FCCA) =1:4;

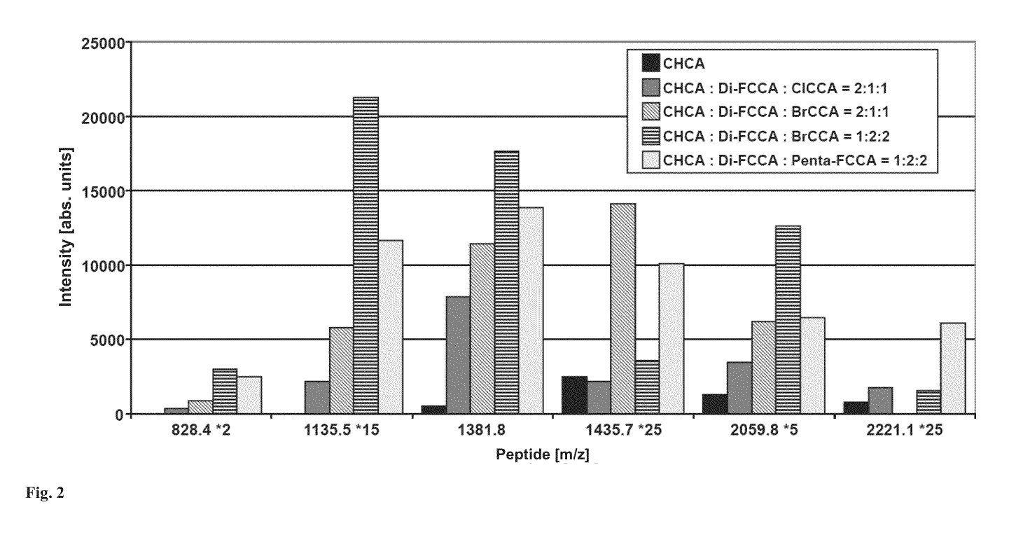

FIG. 2 shows a diagram of the absolute signal intensities of six tryptic 0-casein fragments recorded in the negative ion mode by using CHCA, as well as different ternary matrix systems with participation of matrices applied according to the invention and represented by their respective m/z ratio;

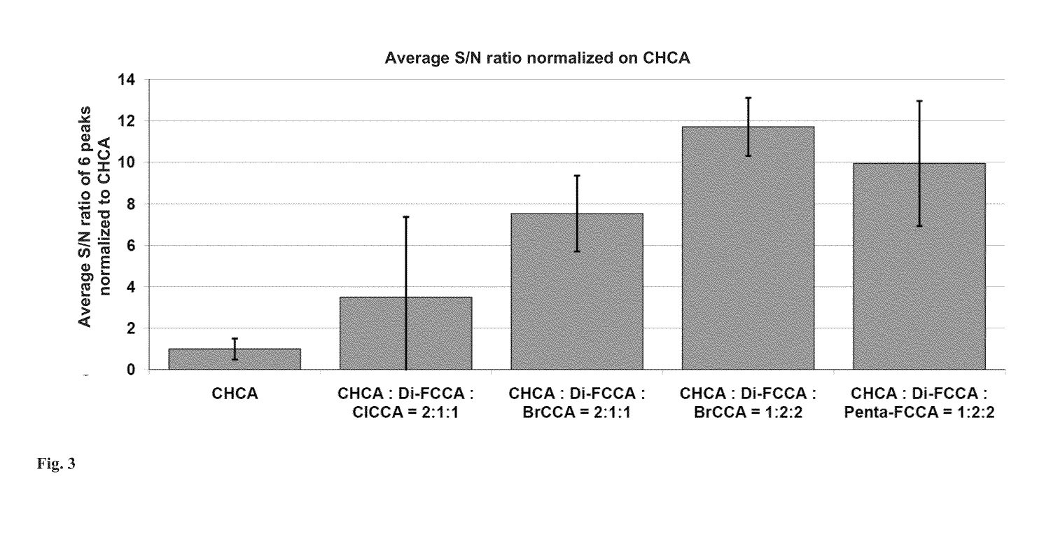

FIG. 3 shows the averaged signal-to-noise (S/N) ratios of all peptides analyzed in FIG. 2 as a function of the used matrix or matrix mixture, respectively, wherein the S/N ratios were normalized on the basis of CHCA;

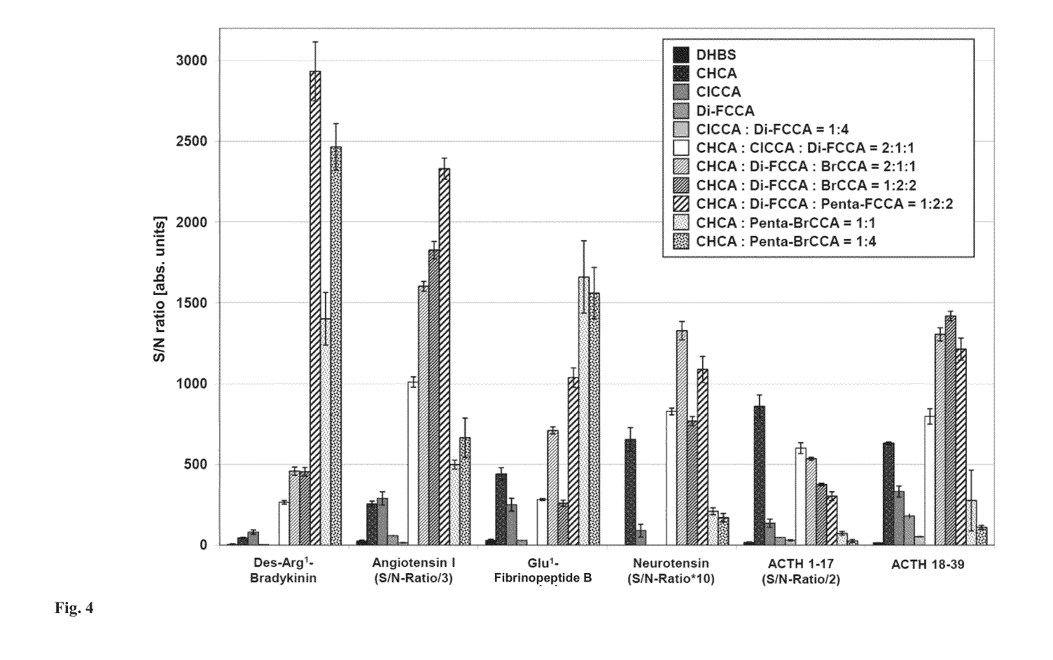

FIG. 4 shows a diagram of the S/N ratios of different conventional matrices and matrix mixtures obtained in the negative ion mode under the use of matrices applied according to the invention, wherein peptides of a standard calibration mixture were used as analyte and the represented measurement values represent the average values from 10 independent individual measurements. For the uniform representation in the diagram, the S/N ratios of the individual analytes were scaled with a constant factor;

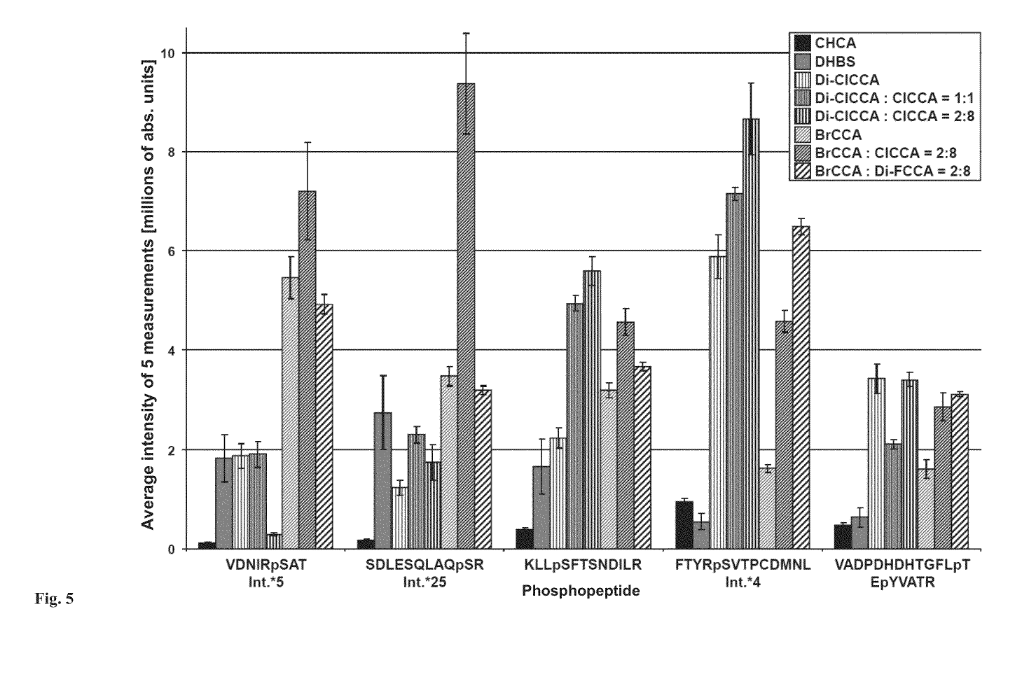

FIG. 5 shows a diagram of the intensities of different phosphopeptides obtained in the negative ion mode and measured with different matrices and matrix mixtures, wherein average values from five independent measurements were applied, and for better clarity the intensities of some phosphopeptides were scaled with a factor, which is constant for all matrices, provided in the abscissa according to the respective peptide sequences;

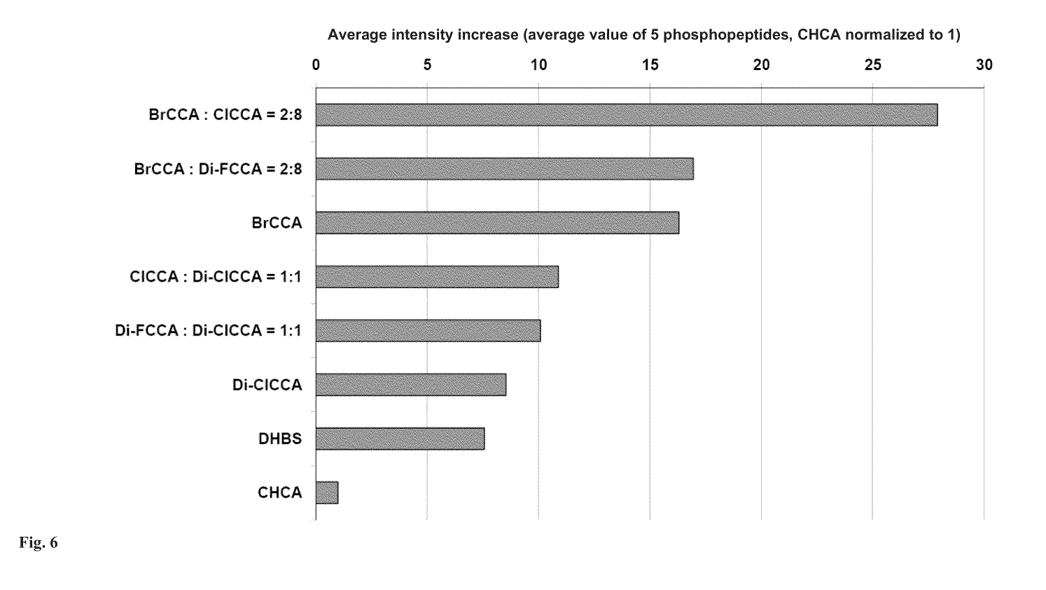

FIG. 6 shows a diagram of the intensities of different phosphopeptides obtained in the negative ion mode by the use of different matrices or matrix mixtures, wherein the signal intensities obtained with the standard CHCA were normalized to 1 after their averaging;

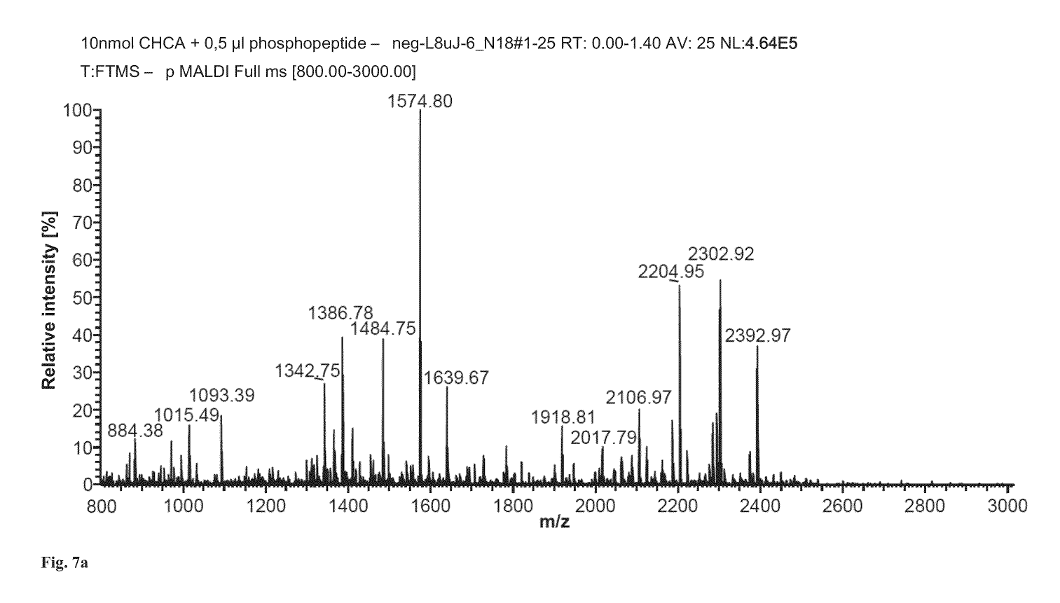

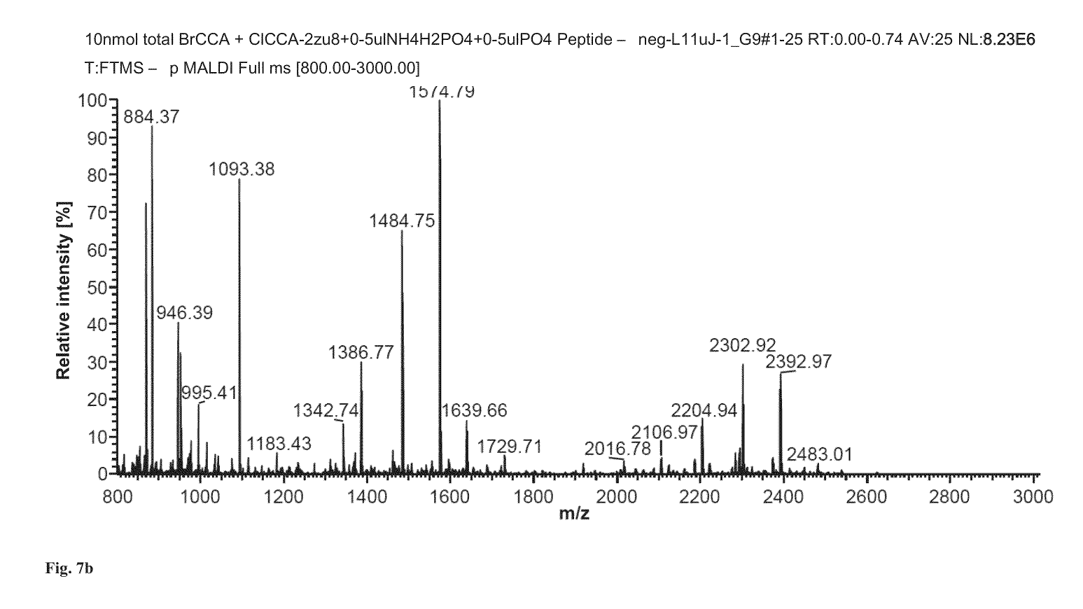

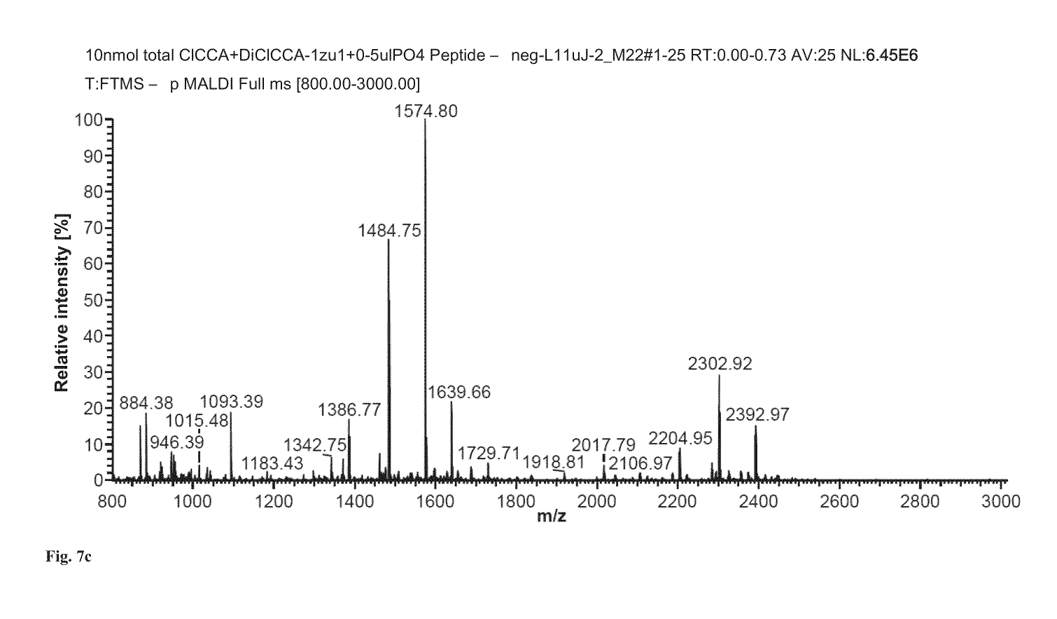

FIG. 7a-c show the mass spectra obtained in the negative ion mode for the detection of different phosphopeptides, with CHCA as a matrix (FIG. 7a), with 4-bromo-α-cyanocinnamic acid (BrCCA):4-chloro-α-cyanocinnamic acid (ClCCA)=2:8 as a matrix (FIG. 7b) and with α-cyano-2,4-dichlorocinnamic acid (Di-ClCCA) : ClCCA=1:1 as a matrix (FIG. 7c); and

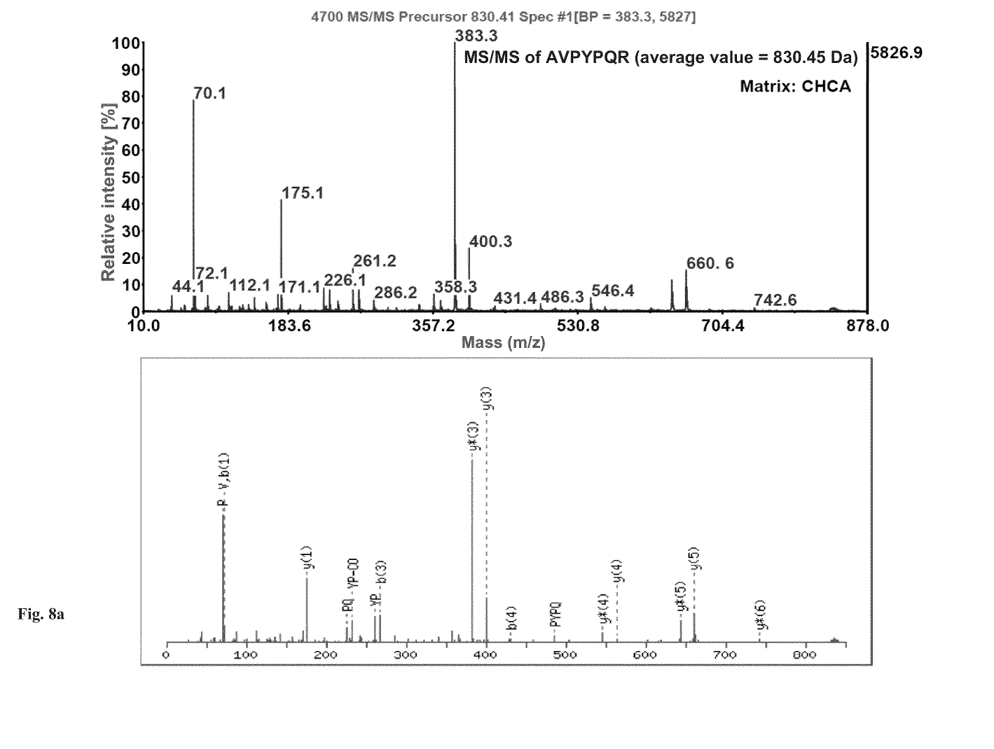

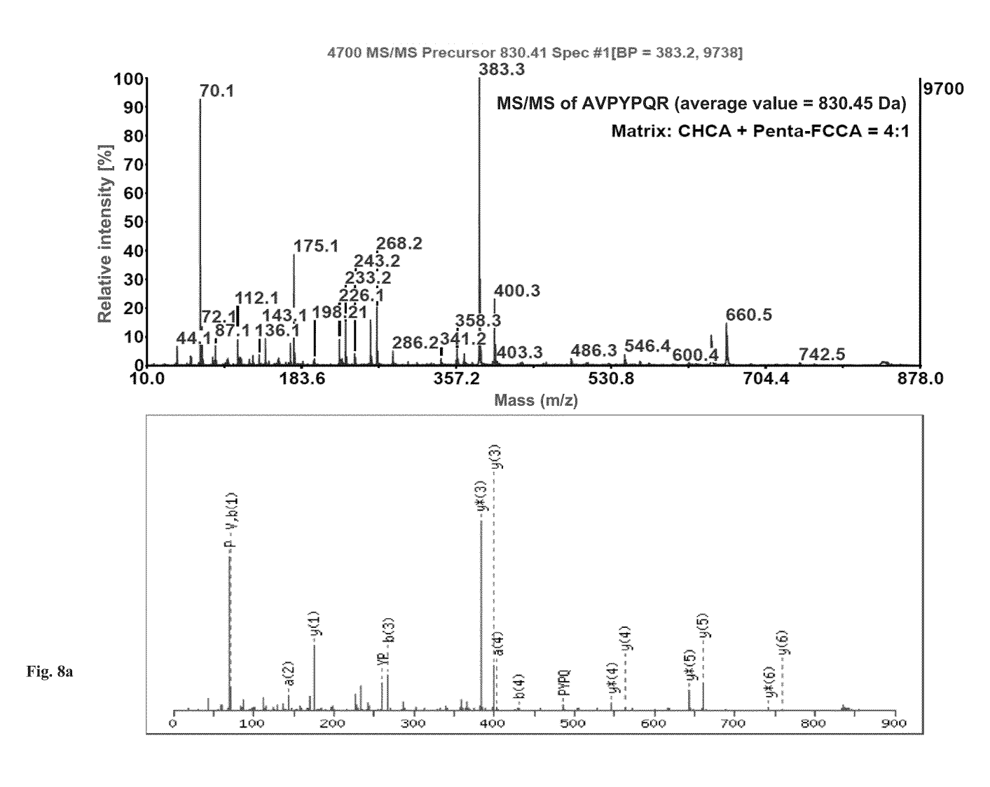

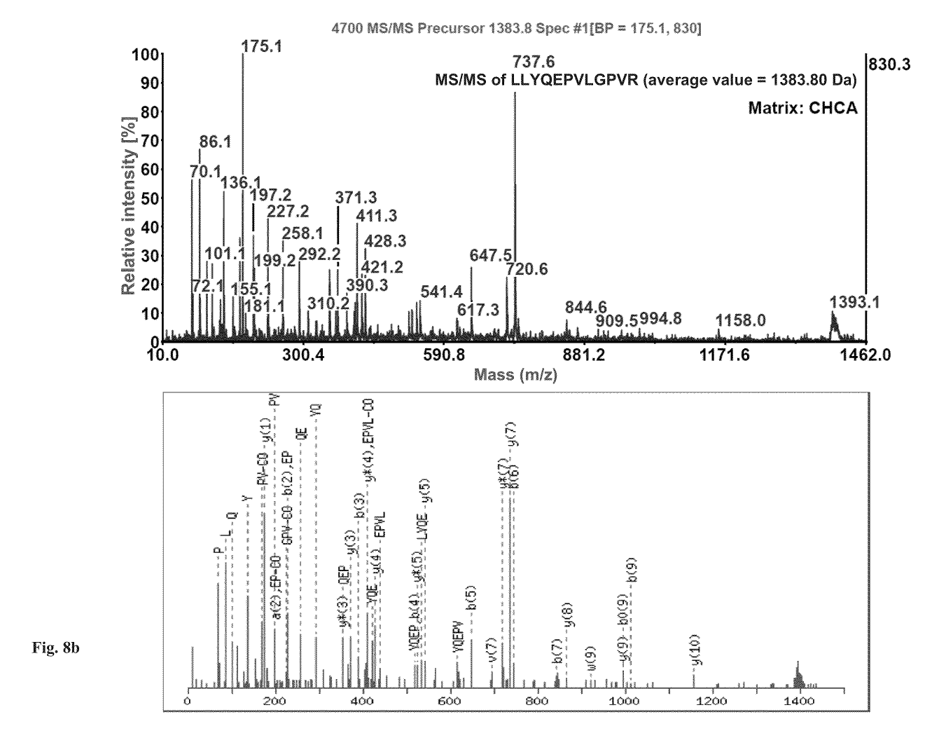

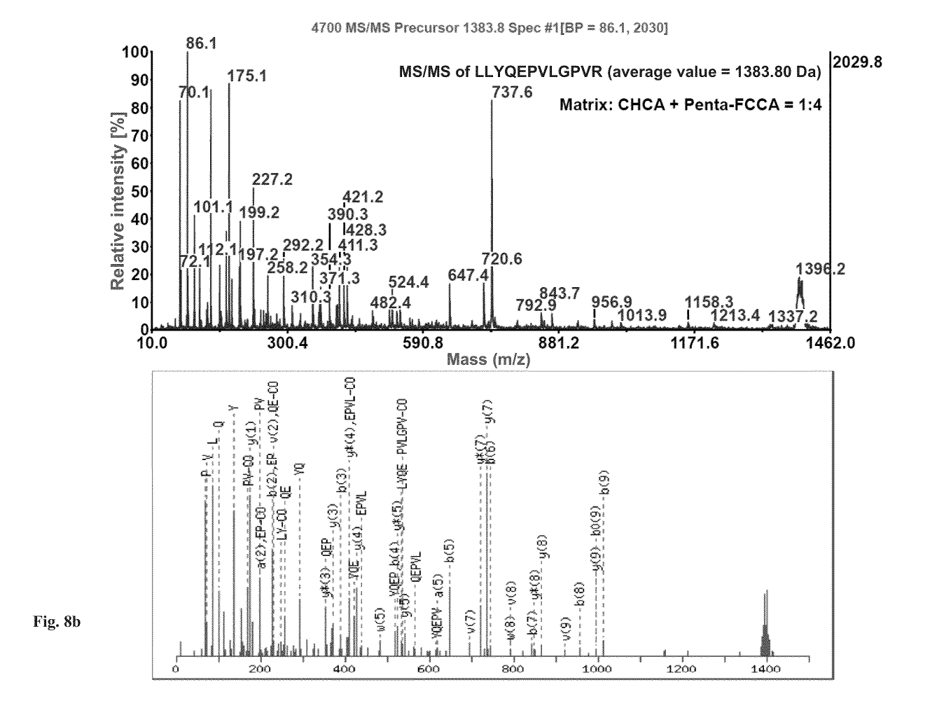

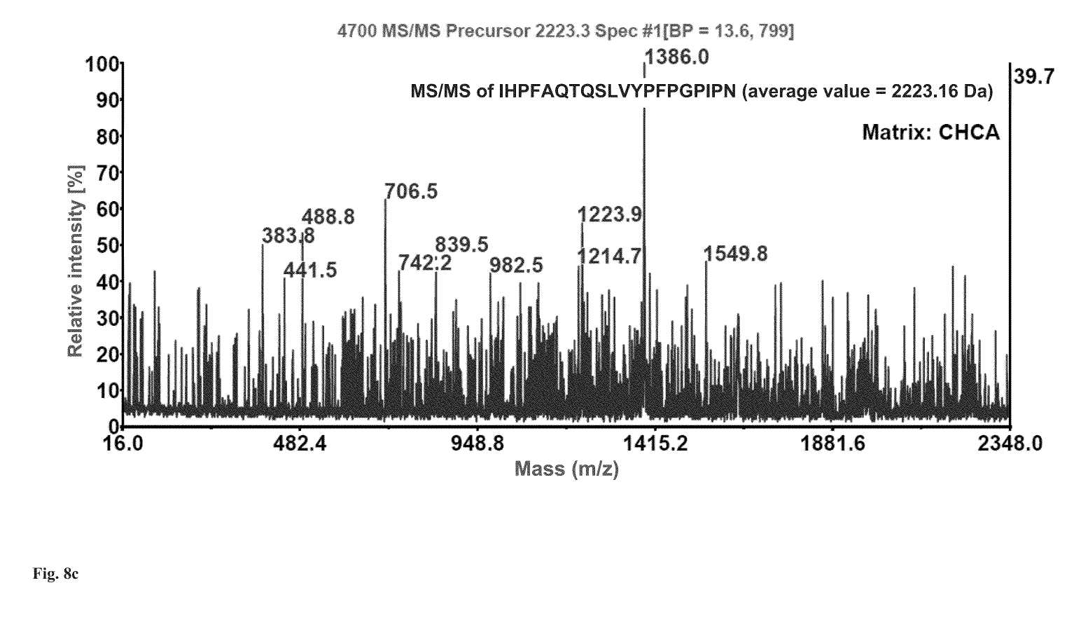

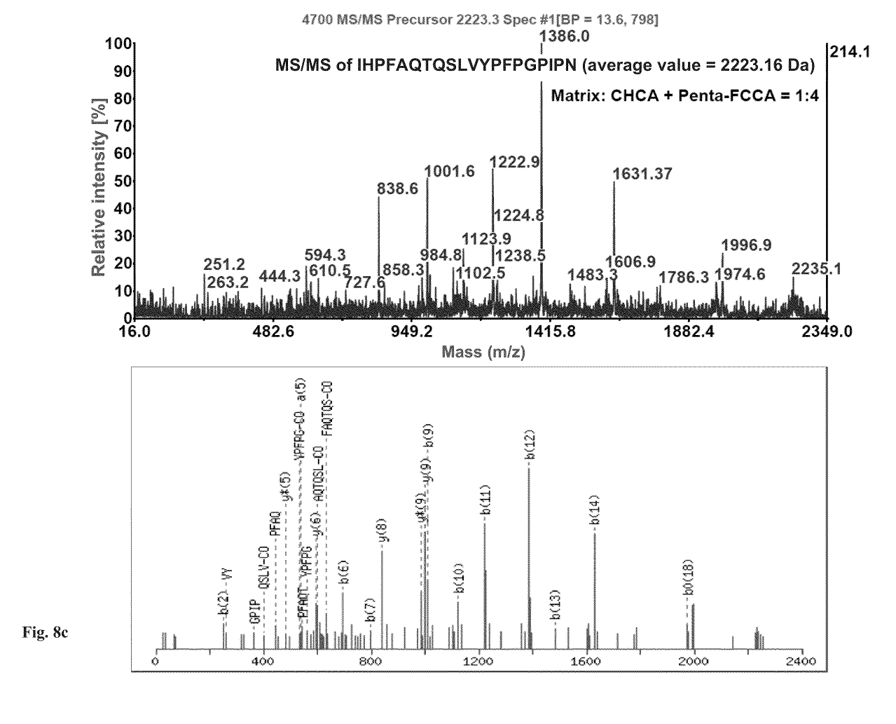

FIG. 8a-c show the fragmentations of different peptides in the positive ion mode.

Synthesis of the substituted α-cyanocinnamic acid derivatives

Halogenated cyanocinnamic acid derivatives used according to the invention can, for example, be obtained by means of condensation of the substituted aldehydes with cyanoacetic acid (derivatives) based on Knoevenagel condensation. The substituted benzaldehydes necessary for that can be obtained in the case of pentahalogen derivatives from the corresponding halogenated toluol derivatives by oxidation with sulfuric trioxide.

Exemplary embodiment for representation of 2,3,4,5,6-pentabromobenzaldehyde 10.1 g (0.02 mol) 2,3,4,5,6-pentabromotoluol are dissolved in 80 g sulfuric trioxide and is flushed with a reflux for 24 hours with exclusion of water. After the reaction has ended, the excess sulfuric trioxide is separated under reduced pressure. The formed dioxonium component is hydrolyzed to aldehyde by adding it to 200 ml of ice. After a brief heating to 75° C. and a subsequent cooling, the aldehyde is filtered, washed to neutral pH value and dried. The recristallization from chlorbenzol gives 8.4 g (0.017 mol) 2,3,4,5,6-pentabromobenzaldehyde as cream-colored pins. Yield: 84% of the theoretical value.

Exemplary embodiment for representation of 2,3,4,5-tetrabromo-α-cyanocinnamic acid 15 g 2,3,4,5-tetrabromobenzaldehyde (MW=421.1 g/mol; 1 equiv.; 3.56 mmol) are flushed with a reflux for 1.5 hours with 317.7 mg cyanoacetic acid (MW=85.1 g/mol; 1.05 equiv.; 3.74 mmol) and 7.8 mg piperidinium acetate (MW=145.2 g/mol; 0.015 equiv.; 0.05 mmol) in 30 ml dry toluol. A water separator serves for separation of the condensation water. After cooling to room temperature, the product is filtered and washed with plenty of cold water.

The raw product is repeatedly recrystallized from a methanol/water mixture. After filtering out and drying in vacuum, 1.65 g α-cyano-2,3,4,5-tetrabromocinnamic acid is obtained. Yield: 95% of the theoretical value.

Exemplary embodiment for representation of α-cyano-2,3,4,5,6-pentafluorocinnamic acid 1 equiv. cyanoacetic acid (n=12.95 mmol; m=1.10 g), 0.9 equiv. 2,3,4,5,6-pentafluorobenzaldehyde (MW=196.07 g/mol; n=11.66 mmol; m=2.287 g) and 0.012 aq piperidinium acetate (n=0.156 mmol, m=22.55 mg) are flushed with a reflux for 3 hours in a sufficient amount of toluol. After cooling to 40° C., the light-yellow crystals are filtered out and washed with cold water. The recrystallization from methanol/water gives yellow oil, which is separated and washed with cold HPLC water, whereupon crystallization takes place. The solving of the crystals in methanol and the precipitation by adding the double volume to cold water causes the product to directly precipitate in crystalline form. Yield: 62% of the theoretical value.

If a certain product does not crystallize during the cooling off after the reaction has ended, it is concentrated under vacuum until the start of the crystallization and the residue is washed with cold water. In order to prevent the oiling out with components such as, for example, α-cyano-2,3,4,5,6-pentafluorocinnamic acid with melting points below the temperature, at which the solubility product has dropped below, the derivative is precipitated in these cases by adding quickly sufficient amounts of cold HPLC water to the clear methanolic solution.

Some derivatives such as, for example, 2,3,6-trichloro-a-cyancinnamic acid or 2,3,4,5-tetrachloro-α-cyancinnamic acid dimerize in the case of too strong energy input to yellow oil with a limited solubility in toluol, which crystallizes in the cold. In order to represent these components, the catalyst portion is increased to 0.03 equiv. and the reaction time is limited to 1 hour; the complete conversion can be tested by means of DC. After cooling off to 50° C., the toluol phase is separated from the oily byproduct and the desired product is precipitated by further cooling. Since the recrystallization is also accompanied by dimerization and yield losses, the raw product is dissolved rapidly in methanol and is quickly precipitated by adding sufficient amounts of cold water.

The cyanocinnamic acid derivatives can be mixed with the analytes by applying a commonly used method in order to prepare a suitable sample for the MALDI mass spectrometry.

An exemplary method for the mixing is, for example, the dried droplet method. The matrix and the analyte are then dissolved and are applied at the same time (by premixing) or one after the other to any desired surface. The crystallization of the matrix with inclusion of the analyte compounds takes place by evaporation of the solvent.

Furthermore, the surface preparation method, in which the matrix or the matrix mixture is solved and applied without analyte on any surface, can be used. The (co-)crystallization of the matrix compound(s) takes place by evaporation of the solvent. The solved analyte is deposited on the crystalline matrix, wherein the analyte compound is included during the recrystallization in a concentrated form by dissolving only the matrix layers which are closer to the surface.

The sublimation method corresponds to the surface preparation method with the difference that the matrix crystallizes not from a solution but is rather deposited on a surface under sublimation by separation from the gas phase.

Finally, a so-called airbrush method is possible, in which the matrix and the analyte are dissolved in a common solvent (mixture) and are distributed by means of dispersion with a spraying device (aerosol formation). Quick evaporation of the solvent takes place through the large surface with the formation of small matrix-analyte crystals. Alternatively, the matrix can be solved also without analyte and sprayed or otherwise applied on surfaces to be examined (for example, tissue sections).

A novel application possibility is the preparation of the matrix as ionic liquid: To this purpose, the solved matrix is mixed and agitated with equimolar amount of base, such as pyridine or diethylamine, whereby a liquid ionic matrix film is formed, which can be applied with the analyte solution on any desired surfaces.

Further below, the term “digestant” means a protein, which has been enzymatically cut out in certain amino acid positions, whereby many small peptides are formed.

An ABI 4800 MALDI TOF/TOF™ analyzer in MS and MS/MS mode at 355 nm as well a MALDI LTQ Orbitrap XL in the Fourier transform mass spectrometry mode and with laser wavelength of 337 nm were used for the performed studies.

The mass spectrometer divides the different ion types (analyte ions, matrix ions) according to their mass/charge ratio. Normally, the ions in MALDI have only one (positive or negative) charge, so that when the charge=1 the mass/charge ratio is equal to the mass of the ions. The abscissa of the spectra gives the mass/charge ratio (and thus in approximately all cases—the mass) of the ions, wherein the unit is dalton or g/mol.

The stronger signals—which are represented by the vertical lines in the spectra, wherein the height of the lines (signals) correlates with the ion species amount which generates this signal—are designated individually with their mass or mass/charge ratio.

The ordinate scale is based on the strongest signal within the respective spectrum and gives the device-dependent absolute value of the strongest signal. All other signals represented in the spectrum are shown in relation to the strongest signal (the left ordinate axis). The right ordinate value is significant only in comparison to the other signals within a given spectrum or other spectra of the same mass spectrometer.

### Example 1:

Increasing the signal intensities

The analyte signal intensities can be increased significantly by the use of more sensitive halogenated matrices. This is illustrated in FIG. 1 for an enzymatic digestant of the protein β-casein with the protease trypsin: The strongest detectable signals in the use of the standard matrix α-cyano-4-hydroxycinnamic acid (CHCA) are due to the analyte-independent matrix own signals (FIG. 1a, top). The peptide signals are clearly visible only in the enlargement (FIG. 1a, bottom). The absolute signal intensity for the strongest fragment amounts to only 439 units. In comparison, the addition of the highly reactive matrix α-cyano-2,3,4,5,6-pentafluorocinnamic acid (penta-FCCA) permits to make considerably more sensitive measurements, see FIG. 1b: The absolute signal intensity of the strongest analyte signal in this case is 9600 units at the same analyte amount; in addition, the analyte signals are clearly better detectable than the matrix own signals in the low mass range.

Parameters:

Mass spectrometer, ABI 4800 MALDI TOF/TOF™analyzer; mode MS; laser wavelength 355 nm; analyte, 1 pmol tryptic β-casein digestant solved in 30% acetonitrile/0.01% trifluoracetic acid; polarity negative; matrix, 10 nmol of the corresponding matrix or matrix mixture solved in 70% acetonitrile at c=20 mM, V=0,5 μl.

### Example 2

Increasing the signal intensities illustrated by ternary matrix mixtures

A mixture of a derivative defined in the claims and two other components as a ternary matrix system can also be used for increasing the sensitivity according to the invention. This is illustrated in FIG. 2 on the basis of the absolute signal intensities of six tryptic β-casein peptide fragments. The protease trypsin used for this purpose showed additional chymotryptic activity, which makes possible, by additional cutting possibilities after, for example, phenylalanine, the generation of the peptide m/z=1381.79 Da (deprotonated) with the sequence (F)LLYQEPVLGPVR(G) or the oxidized peptide (F)LQPEVMGVSKVKEAMAPKHK(E) with m/z=2221.19 Da (deprotonated). The absolute intensities of the respective peptides are represented on the basis of their m/z ratios and depending on the used matrix or matrix mixture. For their representation in a diagram, the signal intensities of the respective peptides were multiplied with a constant factor which is subsequently marked on the abscissa on the basis of the m/z ratio. The used matrix ratios are related to the corresponding substance amount ratios.

It can be clearly seen that when the reference matrix CHCA is used only very low signal intensities can be obtained, see FIG. 2. The ternary matrix mixture CHCA : β-cyano-2,4-difluorcinnamic acid (Di-FCCA) : 4-chlor-β-cyanocinnamic acid (ClCCA)=2:1:1 permits in most cases stronger signal intensities than the pure CHCA matrix. However, the use of 4-brom-β-cyanocinnamic acid (BrCCA) or penta-FCCA for the formation of ternary mixtures makes possible a still clearly more sensitive detection of the analytes.

The increase in the analyte sensitivity by the use of highly halogenated α-cyanocinnamic acid or BrCCA is further clearly seen in FIG. 3, in which the signal-to-sound (S/N) ratios of the peptides analyzed in FIG. 2 are determined and, for the purpose of better comparison, are averaged to an average value and normalized to the CHCA. The S/N ratio shows how many times a signal is stronger than the noise of the spectrum in the respective signal environment and, therefore, it is a measure for the quality of a given signal, since the noise level caused, for example, by the electronic noise of the detector can not be read out only from the signal intensity. As it follows from FIG. 3, the ternary mixtures as matrices according to the invention are clearly more sensitive than the matrices or matrix mixtures used until now.

Parameters:

Mass spectrometer, ABI 4800 MALDI TOF/TOF™analyzer; mode MS; laser wavelength 355 nm; analyte, 1 pmol tryptic β-casein digestant dissolved in 30% acetonitrile/0.01% trifluoracetic acid; polarity negative; matrix, 10 nmol of the corresponding matrix or matrix mixture dissolved in 70% acetonitrile at c=20 mM, V=0.5 μl.

### Example 3

Another example for increasing the analyte sensitivity under the use of a standard peptide calibration mixture is presented in FIG. 4. The measurements were made again in the negative ion mode with different matrices and matrix mixtures and the S/N ratio of the peptides contained in the calibration mixture was measured. The peptides of a standard calibration mixture were used as analytes. A 9:1 (n/n) mixture of 2,5-dihydroxybenzoic acid and 2-hydroxy-5-methoxybenzoic acid and also CHCA, ClCCA, Di-FCCA as well as a mixture of C1CCA and Di-FCCA with and without the addition of CHCA served as references. The BrCCA, penta-FCCA and a-cyano-2,3,4,5,6-pentabromocinnamic acid (penta-BrCCA) were selected as exemplary representatives of the cinnamic acid derivatives applied according to the invention. As it can be seen in FIG. 4, the application of the matrix mixtures according to the invention leads to a clear increase of the sensitivity.

Parameters:

Mass spectrometer, ABI 4800 MALDI TOF/TOF™analyzer; mode MS; laser wavelength 355 nm; Analyte, 0.5 μl Sequazyme Mass Standard Kit (mix 1+2) dissolved in 30% acetonitrile/0.01% trifluoracetic acid; polarity negative; matrix, 10 nmol of the corresponding matrix or matrix mixture dissolved in 70% acetonitrile at c=20 mM, V=0.5 μl, for DHBS c=20 mg/ml in water, V=1 μl.

### Example 4

Increasing the detection intensity of phosphorylated peptides

Due to their easily deprotonable phosphate function, the phosphorylated peptides often show lower ionization efficiency in the positive ion mode than the corresponding non-phosphorylated analytes (Janek et al. “Phosphopeptide analysis by positive and negative ion matrix-assisted laser desorption/ionization mass spectrometry”, Rapid Communication in Mass Spectrometry (2001), Vol. 15, pp. 1593-1599). Therefore, it is recommended to perform the analysis of the phosphopeptides in the negative ion mode, insofar as there is sufficient sensitivity for detection of the analyte(s). FIG. 5 contains a comparison of the use of different conventional matrices as well as matrices according to the invention in relation to the detection sensitivity of exemplary phosphopeptides. The phosphorylation location is marked in the sequences represented on the abscissa in FIG. 5 by a “p” inserted before the corresponding amino acid. The standardly used matrices CHCA and DHBS are contrasted to the derivatives of the α-cyano-2,4-dichlorcinnamic acid (Di-ClCCA) and BrCCA, which were prepared without additional supplements or in the form of binary matrix systems. The pure BrCCA derivative or the one prepared in combination with other matrices permits a clearly more sensitive detection for all analytes. The Di-ClCCA derivative shows, with or without addition of other matrices, particularly high sensitivity for phosphorylated analytes with higher-molecular weight.

Parameters:

Mass spectrometer, MALDI LTQ Orbitrap XL; mode—Fourier-Transform mass spectrometry; laser wavelength 337 nm; analyte, 1 μsynthetic phosphopeptide mix dissolved in 30% acetonitrile/0.01% trifluoracetic acid; polarity negative; matrix, 10 nmol of the corresponding matrix or matrix mixture dissolved in 70% acetonitrile at c=20 mM, V=0.5 μl, for DHBS c=20 mg/ml in water, V=1 μl.

A matrix-specific averaging of the obtained average intensities from FIG. 5 for all phosphopeptides with subsequent normalization on the basis of the CHCA standard permits a quick overview of the efficiency or the sensitivity of the different derivatives and their mixtures and illustrates the strong gain in the analyte signal strength for the newly developed derivatives, see FIG. 6.

Exemplary spectra of the data, on which FIGS. 5 and 6 are based, along with the corresponding increases in the absolute intensities are represented for CHCA (abs. intensity 464,000 units), BrCCA : ClCCA=2:8 (abs. intensity 8,230,000 units) and Di-ClCCA:ClCCA=1:1 (abs. intensity 6,450,000 units) in the spectra in FIG. 7a-c.

### Example 5

Stronger fragmentation in MS/MS analyses

MS/MS analyses serve for structural verification of the analytes. To this purpose, the analyte to be fragmented is isolated through a precursor filter and is then fragmented, for example, by means of collision gas. The fragments formed permit to make statements about the analyte structure, e.g. the amino acid sequence in the peptides as well as possible post-translational possibilities. On the basis of a plurality of possible bond dislocations, a great number of fragments usually appear, whereby the initial total intensity of a given signal is divided into a great number of fragment signals, which is accompanied by clear losses of intensity. Thus, MS/MS spectra often show only weak intensities, which is why many fragments are not detectable or the corresponding MS/MS spectra are completely non-significant in the case of weak precursors. Therefore, it is extremely helpful when higher precursor intensities are obtained through more sensitive matrices and thus subsequently more meaningful fragment spectra can be generated.

The increased fragmentation with more intensive and a greater amount of fragments obtained by the use of penta-FCCA can be seen in FIG. 8a-c. The recorded original spectra contain the measured signal m/z ratios, reproduced by horizontal numerical values; the results of the automatic spectral analyses by the online search engine Mascot (www.matrixscience.com) are represented under the original spectra and contain the annotations of the recognized fragments on the basis of the nomenclature proposed by Johnson, Martin and Biemann (Johnson et al., “Collision-induced fragmentation of (M+H)⁺ ions of peptides. Side chains specific sequence ions”, International Journal of Mass Spectrometry and Ion Processes (1988), Vol. 86, pp. 173-174): Already at 20% addition of penta-FCCA substance amount, strong increases of the fragment signal intensities compared to the reference CHCA can be achieved; see the abs. intensities from FIG. 8 (8a: CHCA, 5827; CHCA+Penta-FCCA, 9700; 8b, CHCA, 830; CHCA+Penta-FCCA, 2030; 8c, CHCA, 40; Penta- FCCA, 214). This permits both the detection of a higher number of fragments (see FIG. 8c) and also of more intensive fragments (see FIG. 8a, b), whereby, on the one hand, the probability for a successful structure clarification increases and, on the other hand, higher intensities in the subsequent MS³ experiments, i.e. further-reaching disintegration analyses of the generated fragments, can be obtained.

In FIG. 8c, due to the low intensity of the obtained signals, no automatic fragment analysis and assignment could be performed for the spectrum (at the very top) obtained with the CHCA reference matrix. In contrast to that, the bottom spectrum in FIG. 8c, obtained with a matrix according to the invention, shows stronger signals, so that an automatic assignment could be performed.

Parameters:

Mass spectrometer, ABI 4800 MALDI TOF/TOF™ analyzer; mode MS/MS; laser wavelength 355 nm; analyte, 1 pmol tryptic β-casein digestant; polarity positive

The features of the inventions disclosed in the above description, in the claims and in the drawings can be essential, both individually and separately, for the implementation of the invention in its different embodiments.

1. Use of a halogenated cyanocinnamic acid derivative with the general formula: 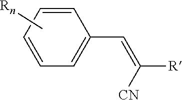
wherein R is selected independently among F, Cl and Br, n=3, 4 or 5 and R′ is selected among COOH, CONH₂, SO₃H and COOR″ with R″ =C₁-C₅-Alkyl; and/or of 4-bromo-α-cyanocinnamic acid and/or of α-cyano-2,4-dichlorcinnamic acid in a matrix for a MALDI mass spectrometry of an analyte.

2. The use according to claim 1, characterized in that the matrix is used for MALDI mass spectrometry of negative ions.

3. The use according to claims 1, characterized in that n=5.

4. The use according to claim 1, characterized in that the matrix for MALDI mass spectrometry is α-cyano-2,3,4,5,6-pentafluorocinnamic acid.

5. The use according to claim 1, characterized in that the matrix for MALDI mass spectrometry is 4-bromo-α-cyanocinnamic acid.

6. The use according to claim 1, where the analyte is selected from the group consisting of protein, peptide, polynucleic acid, lipid, phosphorylated compound, saccharide, medicinal substance, metabolite, synthetic and natural (co)polymer and inorganic compound.

7. The use according to claim 1, where the matrix is mixed with the analyte.

8. The use according to claim 7, characterized in that the molar mix ratio of analyte to matrix is from 1:100 to 1:1000000000, preferably 1:10000.

9. The use according to claim 1, where the matrix further includes at least one other matrix material.

10. The use according to claim 9, characterized in that the at least one other matrix material is selected from the group consisting of α-cyano-4-hydroxycinnamic acid, α-cyano-2,4-difluorocinnamic acid, 2,5-dihydroxybenzoic acid, sinapic acid, ferulic acid, 2-aza-5-thiothymine, 3-hydroxypicolinic acid and 4-chloro-α-cyanocinnamic acid.

11. The use according to claim 1, where the matrix is mixed with an inert filler.

12. The use according to claim 1, where the matrix is available as ionic liquid.

13. A matrix for a MALDI mass spectrometry of an analyte, which is a halogenated cyanocinnamic acid derivative with the general formula: 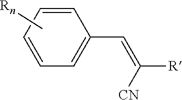
wherein R is selected independently among F, Cl and Br, n=3, 4 or 5 and R′ is selected among COOH, CONH₂, SO₃H and COOR″ with R″ =C₁-C₅-alkyl; and/or comprises 4-bromo-α-cyanocinnamic acid and/or α-cyano-2,4-dichlorocinnamic acid.

14. A method for analyzing an analyte, the method comprising:
combining the analyte with a matrix material, where the matrix material includes a halogentated cyanocinnamic acid derivative with the general formula: 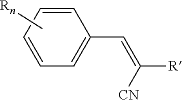
wherein R is selected independently among F, Cl and Br, n=3, 4 or 5 and R′ is selected among COOH, CONH₂, SO₃H and COOR″ with R″ =C₁-C₅-Alkyl; and/or of 4-bromo-α-cyanocinnamic acid and/or of α-cyano-2,4-dichlorcinnamic acid; to form a co-crystallized sample; and
analyzing the co-crystallized sample using matrix-assisted laser desorption/ionization techniques.
